(19) United States
(12) Patent Application Publication
(10) Pub. No.: US 2008/0208045 A1
(43) Pub. Date: Aug. 28, 2008

(54) Optimization of User Settings for an Ultrasonic Imaging System

(75) Inventor: Matthew Rielly, Seattle, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/913,777

(22) PCT Filed: May 3, 2006
(86) PCT No.: PCT/IB06/51380

§ 371 Date: Nov. 7, 2007

Related U.S. Application Data
(60) Provisional application No. 60/679,639, filed on May 10, 2005.

(51) Int. Cl. A61B 8/00; G01S 7/52

(52) U.S. Cl. 600/437

(57) ABSTRACT

An apparatus and method are described for developing system presets for imaging procedures performed by an ultrasound system. A plurality of ultrasound systems each compile a log of system preset settings used by system operators in diagnostic exams. Periodically these logs are transmitted to a service center where the system settings data is aggregated into a common database. The data of the database is analyzed to develop factory installed system presets derived from a large number of ultrasound system users over a wide geographic area. Specialized presets can be developed for regional or local groups of users.

This invention relates to medical diagnostic ultrasound systems and, in particular, to adjustable user setting for a diagnostic examination.

Ultrasound systems have traditionally been equipped with a wide variety of user controls and control settings which enable the system operator to adjust and optimize the performance of the ultrasound system for a particular diagnostic examination. The operator generally begins by picking one of several probes available on the system. Ultrasound probes can differ in the frequency of operation, with higher frequencies used to image at shallow depths with good image clarity and lower frequencies needed at greater abdominal depths but with lesser image quality. Probes can differ in the size of the imaging aperture, the number and pitch of the elements of the transducer array, and the density of the scanlines transmitted and received. Astute selection of these factors can provide the best images for a particular type of imaging exam.

System setting can also affect the quality of an image in a given exam. The power level at which the probe elements are driven will control the depth to which the ultrasonic waves will penetrate, facilitating better images at greater depths. The transmitted beams can be focused at one or more focal zones of a selected depth or depths. During echo reception the time gain control (TGC) and overall gain by which the echoes are amplified will affect the signal to noise characteristic of the image. Setting the ensemble length and color box size will affect the precision and frame rate during spectral and color Doppler imaging. Filter settings such as wall filter parameters will control the amount of clutter and noise in the image. Image processing settings such as interpolation, spatial and frequency compounding, and the RES setting will affect the speckle noise and smoothness of tissue in the image. A clinician can thus spend a considerable amount of time optimizing these and many other settings for the best images in a given diagnostic exam.

Mindful of this difficulty, ultrasound manufacturers have taken steps to expedite this process. All ultrasound systems today provide the ability to store the settings for an examination. After a clinician has optimized the settings for an exam which is conducted routinely, the clinician can save the settings on the system so that they can be recalled when the same diagnostic exam is performed in the future. When systems are networked the exam settings saved on one ultrasound system can be accessed from another ultrasound system on the network and used for exams on the second machine as described in U.S. Pat. No. 5,891,035 (Wood et al.) System presets can be sent to ultrasound systems at remote locations by electronic messaging as described in U.S. Pat. No. 5,897,498 (Canfield, II et al.) Additionally, ultrasound manufacturers provide factory-installed system presets matched to specific exam types which can be performed by the system. The Tissue Specific Imaging (TSI) feature on Philips Medical Systems ultrasound machines will automatically set up selected presets for a particular type of exam after the clinician has chosen the exam type from a menu of exams which the system can perform. The presets are determined by observing the settings most commonly used by clinicians during specific types of exams. However, the system presets are only representative of the preferences of the users who were observed and whose settings were recorded. The presets may not be representative of those preferred by other groups of users or of the universe of system users in general. It is desirable to refine the process for obtaining such presets so that they are better representative of a broader range of system users. It would also be desirable to be able to develop presets tailored for specific groups of users. For instance, the patients in one region may be bigger or heavier in general than patients in another region, calling for different preferred presets in the different regions.

In accordance with the principles of the present invention, a diagnostic ultrasound system and method are described which enable an ultrasound system to be configured with a more refined selection of user presets. An ultrasound machine captures the settings for exams performed on the system. Periodically the captured settings of this and other ultrasound systems are transmitted to a service center or factory where presets are prepared for ultrasound systems. The received information is analyzed to determine a broadly-based set of system preset settings which can be used in existing and new systems. This analysis can be performed on system settings from a select group of ultrasound systems or ultrasound systems in a particular geographic region to tailor system presets designed for that particular group or region.

In the drawings:

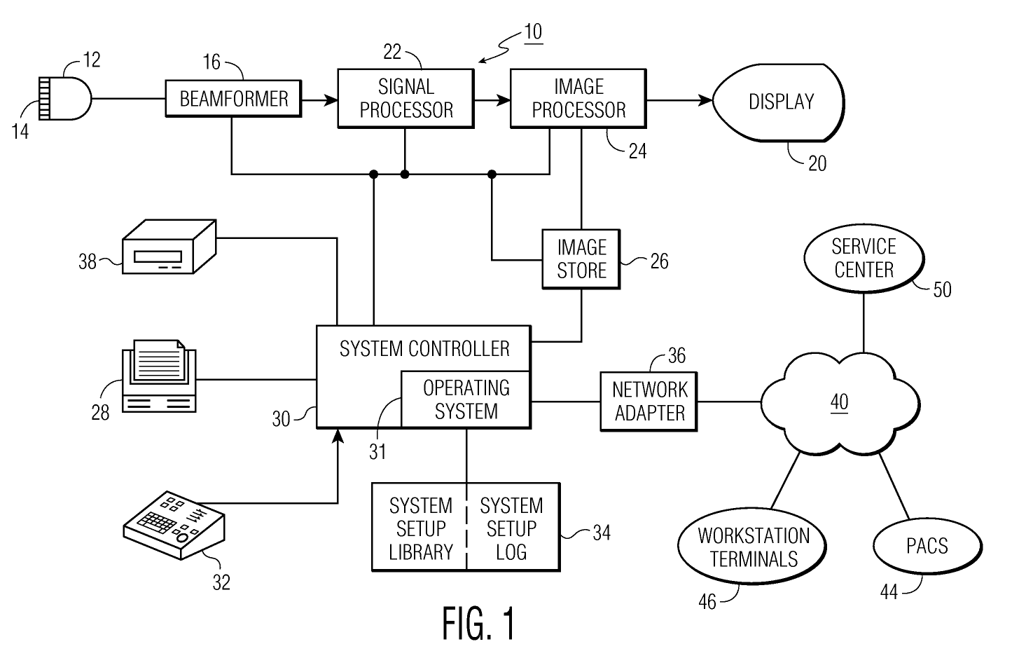

FIG. 1 illustrates in block diagram form an ultrasonic diagnostic imaging system constructed in accordance with the principles of the present invention.

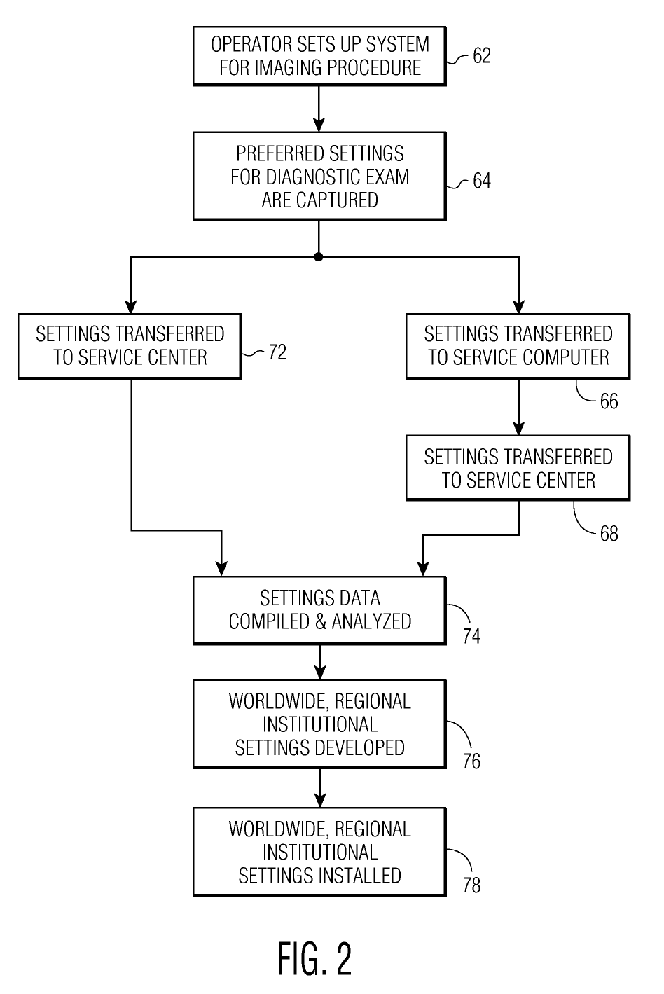

FIG. 2 is a flowchart of a method for developing a broadly-based set of ultrasound system presets.

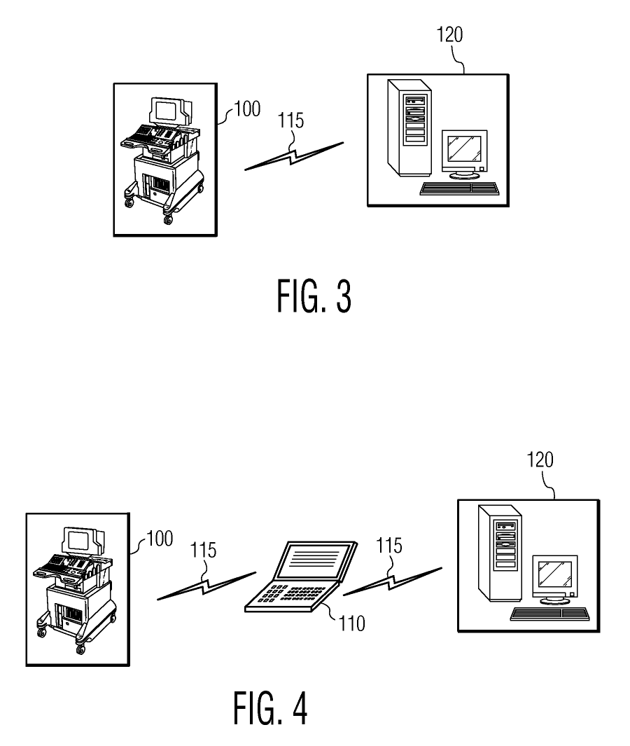

FIG. 3 illustrates one technique for transmitting ultrasound system settings to a service center for analysis.

FIG. 4 illustrates a second technique for transmitting ultrasound system settings to a service center for analysis.

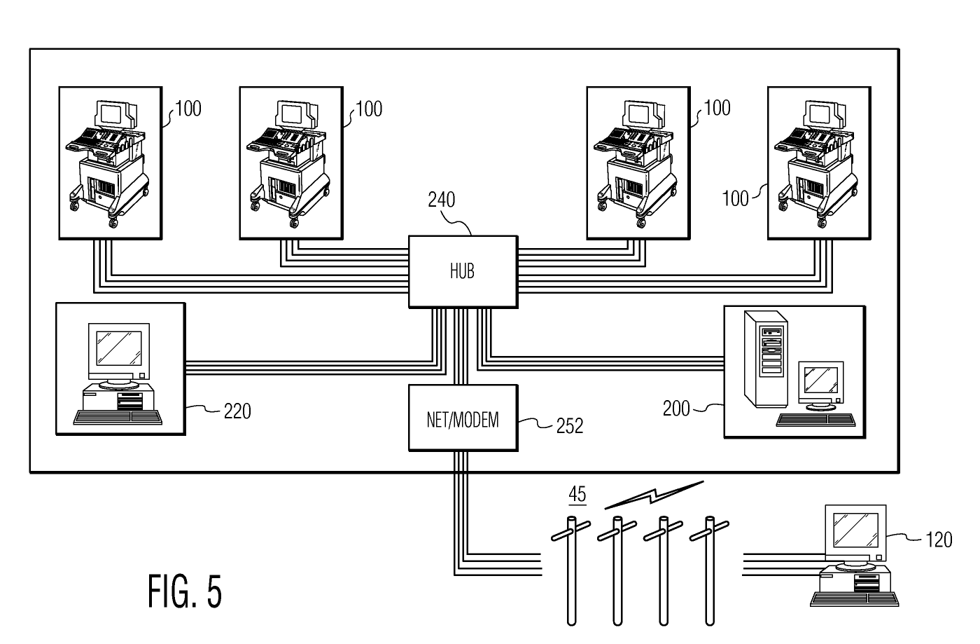

FIG. 5 illustrates a group of ultrasound systems for which specially tailored system presets are developed.

Referring first to FIG. 1, an ultrasonic diagnostic imaging system 10 and network constructed in accordance with the principles of the present invention is shown in block diagram form. The ultrasound image acquisition, processing and display path of the ultrasound system 10 starts with an array probe 12 having an array of transducer elements 14. The transducer array transmits ultrasonic waves under control of a beamformer 16 and receives echo signals from the subject being imaged which are converted to electrical signals. The signals received by the individual elements of the array are appropriately delayed and combined by the beamformer 16 to form coherent echo signals. The echo signals may then undergo specific processing for the type of information acquired and to be displayed such as detection, filtering, Doppler processing, harmonic signal separation, and the like. This processing is performed by a signal processor 22. The processed signals are formed into an image of a desired display format by an image processor 24 and the processed images are displayed on an image display 20. The images may be stored in an image store 26 for further processing and review or later display.

The functioning of the processes of the image acquisition, processing and display path is controlled and coordinated by a system controller 30 which is coupled to the components of the signal path. The system controller responds to commands from a user which can be input by a graphical user interface on a display or from a control panel 32 or voice recognition system. The system controller runs an operating system (OS) 31 which performs functions involving the user interface and/or the display 20 and communications with peripheral devices such as a printer 28 and a video recorder 38. The OS also controls communication over a network 40 through a network adapter 36 such as an Ethernet card or modem. The network 40 can be of various types such as Ethernet, FDDI, PPP, token-ring, IEEE 802.11, I²C and others. When the ultrasound system is connected to the network 40 it can communicate with other devices on the network, examples of which include picture archival and communication systems (PACS) 44, and workstation terminals 46.

The system controller 30 communicates with system data store 34. Located on the system data store is a library of system setup parameters for different types of diagnostic exams. An appropriate set of setup parameters is accessed by the system controller and initially used for a diagnostic imaging procedure depending on the type of procedure selected by the clinician operating the ultrasound system. For instance, the clinician may connect a phased array probe to the ultrasound system and select a cardiac exam. The system controller responds by selecting system setups for a cardiac exam including such parameters as Doppler mode initiation, Doppler ensemble length, wall filter values, and harmonic imaging. The focal depth would be set to that expected for the heart of an average adult. TGC and gain values are also preset. The clinician will generally find most of these values to be acceptable and will then adjust the controls on the control panel 32 and graphic user interface for the desired image of the particular patient being examined. If the clinician connects a linear array probe to the system 10 for a fetal exam, a different set of presets would be retrieved for the different imaging depth and probe type used for the fetal exam. Most ultrasound systems today are installed with a system setup library of sets of preset values for typical exam types.

In accordance with the principles of the present invention the system data store 34 also includes a storage area for a system setup log. The system setup log is a part of the ultrasound system where sets of imaging procedure settings used by the clinician are stored. Entries in this log may be made manually by the clinician, automatically by the ultrasound system, or both. For instance, the clinician may select a particular exam type and then observe the imaging performance with a set of factory installed presets recalled from the system setup library. The clinician may have a preference for adjustment to a number of the preset settings and make those adjustments. If the clinician finds the adjusted settings to be to his or her liking the clinician may want to save the settings for future exams. The clinician accesses the save control for system settings, gives the set of settings a title such as “Dr. Smith Cardiac Settings,” and saves the set of customized system settings in the system setup library. The set of customized settings is concurrently saved in the system setup log.

Alternatively or additionally, system settings may be automatically saved in the system setup log by the system controller. It is desirable not to save settings that are nondiagnostic or not favored by the clinician. For example, system settings should not be saved while the clinician is experimenting with a set of settings and making adjustments. Several different cues can be used to identify settings that a clinician favors. One technique is to save a group of settings only after they have been active and unchanged for a significant period of time. If the set of settings has not been adjusted for awhile and has been in use for an extended period of time, it may be assumed that the clinician is pleased with them and hence they can be saved in the system setup log. Another technique is to cue off of an action such as saving an image. When the clinician finds what is believed to be an image useful for the diagnosis the clinician may hit the “Print” button on the control panel 32 to send the image to the printer 28. The actuation of the Print button can also be taken as a cue to save the system settings in the system setup log. Another cue can be the actuation of the video recorder 38. When the clinician hits the “Record” button on the control panel to begin recording a sequence of realtime images, the system controller can automatically save the system settings in the system setup log.

Other useful contextual information is also saved with the set of system settings stored. The type of exam may be saved, although often the type of exam will be apparent from the settings themselves. The date and time at which the settings were saved may also be recorded. A clinician identifier may also be saved, the usefulness of which will be discussed below. An indication of whether the settings are for the same patient and/or the same exam as the previously stored set of settings may be saved. This enables settings stored from the same exam with the same patient to be recognized as duplications. On the other hand, a recording of the same set of settings with different patients indicates that the set of settings is of great favor with the clinician and may be a candidate for a factory setting. Personal patient information such as the name of the patient is not needed and should not be saved. Information which might compromise patient privacy should be avoided and in many instances alternative information can serve the desired purpose such as recording a yes/no indicator of whether this is a different patient than that of the previous set of saved settings.

With the consent of the ultrasound system owner or user the sets of settings recorded in the system setup log are periodically transferred to a service center or manufacturer facility where they are added to a database of settings from other ultrasound systems and used to refine the factory preset settings for this and/or other ultrasound systems. This transfer can be done manually by transporting a data medium such as a disk with the setup log data on it back to the service center or other facility. In the embodiment of FIG. 1 the transfer can be done electronically by communicating the setup log data to the service center 50 over the network 40. Periodically the operator or a serviceperson can send the setup log data over the network to the service center. Alternatively, the operator or owner of the ultrasound system may make arrangements to have the data accessed or sent in response to an electronic request from the service center over the network 40, such as at nighttime when the ultrasound system is not in use. By these means useful sets of system settings can be gathered from a significant installed base of ultrasound systems dispersed across a country or even across the world. The sets of system settings are aggregated in a common database and analyzed to determine whether particular sets of settings are more commonly used than others. Sets of system settings which are favored by a significant percentage of users can be identified and used as factory settings in the current installed base or in ultrasound systems manufactured in the future.

A method for carrying out this entire procedure is illustrated in FIG. 2. The process begins at 62 when an ultrasound system operator sets up an ultrasound system for an imaging procedure. After a set of settings have been identified as being preferred for the exam, some techniques for doing so being described above, the set of settings is captured at 64 and stored in the system setup log. The setup log data is transferred to a service center in steps 66, 68, 72. On the left side of the flowchart at 72 the settings are transferred directly to the service center. One way for doing this is illustrated in FIG. 3. In this illustration the system settings data is communicated electronically (indicated at 115) from an ultrasound system 100 to a database 120 at the service center. Another way to transfer the information is to transfer it from the ultrasound system 100 to a portable device 110 such as a serviceperson's computer shown in FIG. 4. The data is subsequently transferred from the portable device 110 to the service center database 120. A suitable portable device is described in International Pub. No. WO 01/70100 filed by Koninklijke Philips Electronics N.V. As described therein, service personnel carry diagnostics computers which can download data from ultrasound systems, either at the site of the ultrasound system or over a network from a remote location. The ultrasound system information is thereafter uploaded to a central diagnostics location such as a service center or the manufacturing or design location of the ultrasound system manufacturer.

Continuing with the flowchart of FIG. 2, the settings data from multiple ultrasound systems are compiled into a database and then analyzed at 74 to identify popular sets of settings or, where a wide range of setting are found to be used, a nominal set of settings. These identified sets of settings are then used directly or indirectly to develop factory presets for current or new ultrasound systems at 76. The developed sets of settings can be developed from a worldwide settings database and applied worldwide at 8, or developed from a regional or smaller database and applied on a less widespread basis at 78. For instance, the geographic region from which a settings log came can be saved with the settings data. A database of a particular geographic region can then be compiled. For instance, all of the settings log data from a particular country can be aggregated into one database. Patterns, trends, averages and derivations of these settings from a common geography can then be used to develop factory presets for ultrasound systems shipped to users in that country. The factory settings will thus be tailored for the most useful settings for the patients and system operators in that country.

Even more precisely defined preferred settings can be developed for smaller groups of users as illustrated by FIG. 5. In this illustration a number of ultrasound systems 100 of a hospital or regional hospital organization are networked together by a hub 240 and a network server 200. Sonographers may move around from hospital to hospital within the hospital chain, storing and recalling their preferred system settings through the server 200. Workstations 220 where images are processed and read may also be on the network. The network is connected externally by a net/modem 252 by which system setup logs from all of the ultrasound systems 100 are communicated either by wire or wirelessly as indicated at 45 to the service center database 120. The system setup logs from only the ultrasound systems of this hospital or group of hospitals is aggregated in one database and analyzed, enabling system setups to be identified and developed which uniquely reflect the setting values preferred at this regional institution. In this way the ultrasound system manufacturer can manufacture and deliver ultrasound system with diagnostic procedure setups specially tailored for a specific group of ultrasound system users.

What is claimed is:

1. An ultrasonic diagnostic imaging system development tool comprising:
a plurality of ultrasound systems, each of which records sets of system settings which are favored by users;
means for transferring the records of system settings from the plurality of ultrasound systems to a common database; and
means for utilizing the database to develop system presets for an ultrasound system.

2. The ultrasonic diagnostic imaging system development tool of claim 1, wherein certain ones of the ultrasound systems include a user control which is actuated by a user to record system settings.

3. The ultrasonic diagnostic imaging system development tool of claim 1, wherein certain ones of the ultrasound systems include means for automatically recording system setting upon the occurrence of a predetermined event.

4. The ultrasonic diagnostic imaging system development tool of claim 3, wherein the predetermined event comprises the use of a set of system settings for a predetermined period of time.

5. The ultrasonic diagnostic imaging system development tool of claim 4, wherein the predetermined event comprises saving data from a diagnostic exam.

6. The ultrasonic diagnostic imaging system development tool of claim 1, wherein the means for transferring comprises a data network.

7. The ultrasonic diagnostic imaging system development tool of claim 1, wherein the means for transferring comprises a portable data medium.

8. The ultrasonic diagnostic imaging system development tool of claim 7, wherein the portable data medium comprises a service computer.

9. The ultrasonic diagnostic imaging system development tool of claim 1, wherein the means for utilizing comprises a service center which produces system preset data for an ultrasound system.

10. The ultrasonic diagnostic imaging system development tool of claim 1, wherein the system settings include the type of diagnostic exam for which the system settings are used.

11. A method for developing factory presets for an ultrasound system comprising:
recording system settings employed by users of a plurality of ultrasound systems;
transferring the recorded system settings to a common database;
analyzing data of the system settings database to determine a set of system presets for an ultrasound system.

12. The method of claim 11, wherein recording further comprises recording system setting employed by users of a plurality of ultrasound systems of a common medical facility or medical facility network; and
wherein analyzing further comprises determining a set of system presets for ultrasound systems of the common medical facility or medical facility network.

13. The method of claim 11, wherein recording further comprises recording the type of diagnostic exam for which the system settings were employed.

14. The method of claim 11, wherein recording further comprises manually recording system settings.

15. The method of claim 11, wherein recording further comprises automatically recording system settings.

16. The method of claim 11, wherein transferring further comprises transferring recorded system settings over a network.

17. The method of claim 11, wherein transferring further comprises:
transferring recorded system settings to a transportable data medium; and
transferring recorded system settings from the transportable data medium to the common database.

18. The method of claim 11, wherein analyzing further comprises analyzing data of the system settings used in a predetermined country or geographic region.

19. The method of claim 11, wherein analyzing further comprises determining a set of system presets for an ultrasound system of the type for which system settings were recorded.

20. The method of claim 11, wherein analyzing further comprises determining a set of system presets for a newly developed ultrasound system.
